(19) United States
(12) Patent Application Publication
(10) Pub. No.: US 2014/0170248 A1
(43) Pub. Date: Jun. 19, 2014

(54) MUSCLE CRAMP RELIEF LOTION

(71) Applicant: Mohamed H. Tarifi, Hillsboro Beach, FL (US)

(72) Inventor: Mohamed H. Tarifi, Hillsboro Beach, FL (US)

(21) Appl. No.: 13/719,223

(22) Filed: Dec. 18, 2012

(51) Int. Cl. A61K 36/899; A61K 36/53; A61K 36/575; A61K 36/355; A61K 36/63; A61K 36/48

(52) U.S. Cl. CPC A61K 36/899; A61K 36/63; A61K 36/48; A61K 36/575; A61K 36/355; A61K 36/53
USPC 424/745

(57) ABSTRACT

Seventy percent of Americans over 50 years old experience painful muscle cramps. Athletes, professional and amateurs suffer from muscle cramps every day. Worst muscle cramps occur at night while people are sleeping, resulting in loss of sleep and productivity. A formulation comprising deionized water, ethyl alcohol and six oil extracts including jasmine, sweet pea, magnolia, lavender, meadow and honey suckle has been proven to relief muscle cramps safely, effectively and instantly.

### BACKGROUND OF THE INVENTION

The present invention relates to muscle cramp relief lotions and, more particularly, to a topically applied lotion that will instantly relieve muscle cramps.

Muscle cramps can happen unexpectedly and can be quite painful. Often, the longer the muscle cramp lasts, the longer the residual pain associated with the muscle cramp lasts. Athletes can get a muscle cramp that can cause their removal from an athletic event. It is often important for the athlete to resume play without delay. It has been reported that 70 percent of American over 50 years old experience muscle cramps.

While there are muscle cramp relief formulations currently on the market, some of which are topical and others are ingested, these formulations often take time to provide relief, which results in considerable pain and loss of sleep and productivity.

As can be seen, there is a need for a fast and effective formulation and method for alleviating muscle cramps.

### SUMMARY OF THE INVENTION

In one aspect of the present invention, a formulation comprises deionized water; ethyl alcohol; and six oil extracts consisting of jasmine oil extract, sweet pea oil extract, magnolia oil extract; lavender oil extract, meadow oil extract and honey suckle oil extract.

In another aspect of the present invention, a formulation comprises from about 60 to about 75 percent deionized water; from about 20 to about 30 percent ethyl alcohol; and from about 6 to 10 percent oil extracts of jasmine, sweet pea, magnolia; lavender, meadow and honey suckle.

In a further aspect of the present invention, a method for treating muscle cramps comprising topically administering an effective amount of a formulation comprises deionized water; ethyl alcohol; and oil extracts of jasmine, sweet pea, magnolia; lavender, meadow and honey suckle.

These and other features, aspects and advantages of the present invention will become better understood with reference to the following description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a formulation for relieving muscle cramps. The formulation can be provided as a topical lotion or a toilette that can be applied to the site of the muscle cramp. The formulation can quickly, safely, effectively and instantly alleviate muscle cramps. The formulation includes a unique combination of oil extracts in an alcohol and water solution.

The formulation of the present invention includes a unique combination of oil extracts in alcohol. While not being limited to any particular mechanism of action, the formulation of the present invention can act as an effective vasodilator. As the blood vessels dilate, they allow more blood to reach the cramped muscle, providing the muscle with appropriate nutrients that will instantly relieve cramps. The formulation of the present invention can be similarly as effective as an intravenous of electrolytes, which is proven to stop cramps instantly.

The formulation of the present invention can include from about 20 to about 30 percent (v/v) of ethyl alcohol, typically about 25 percent ethyl alcohol, and from about 6 to about 10 percent (v/v) oil extracts, as described below. The remainder of the formulation includes deionized water, typically from about 65 to about 75 percent (v/v) thereof. The formulation can include various adjuncts to form the formulation into a particular media. For example, the formulation can include adjuncts to form the formulation into a lotion, a cream, a liquid for absorption onto a toilette, or the like.

The oil extracts can include from 6 to about 10 percent of the following oil extracts: jasmine, sweet pea, magnolia, lavender, meadow and honey suckle In some embodiments, equal amounts of each of the above oil extracts can be used in the formulation. In some embodiments, about 1 percent of each of the above oil extracts can be used in the formulation.

It should be understood, of course, that the foregoing relates to exemplary embodiments of the invention and that modifications may be made without departing from the spirit and scope of the invention as set forth in the following claims.

What is claimed is:

1. A formulation comprising:
Deionized water;
ethyl alcohol; and
oil extracts including jasmine, sweet pea, magnolia; lavender, meadow and honey suckle.

2. The formulation of claim 1, wherein the oil extracts are 6-10 percent of the formulation by volume.

3. The formulation of claim 1, wherein deionized water is from about 60 to about 75 percent of the formulation by volume.

4. The formulation of claim 1, wherein ethyl alcohol is from about 20 to about 30 percent of the formulation by volume.

5. The formulation of claim 1, wherein the oil extracts include at least 1 percent each by volume of oil extracts of jasmine, sweet pea, magnolia, lavender, meadow and honey suckle.

6. The formulation of claim 5, wherein each of the oil extracts are present in equal volume in the formulation.

7. A formulation comprising:
from about 60 to about 75 percent deionized water;
from about 20 to about 30 percent ethyl alcohol; and
from about 6 to about 10 percent oil extracts, wherein the oil extracts include jasmine, sweet pea, magnolia, lavender, meadow and honey suckle

8. The formulation of claim 7, where deionized water is present in about 69 percent by volume.

9. The formulation of claim 7, wherein ethyl alcohol is present in about 25 percent by volume.

10. A method for treating muscle cramps comprising topically administering an effective amount of a formulation, the formulation comprising:
Deionized water;
ethyl alcohol; and
oil extracts including jasmine, sweet pea, magnolia; lavender t, meadow and honey suckle.

11. The method of claim 10, wherein the oil extracts consisting of jasmine, sweet pea, magnolia, lavender, meadow and honey suckle are included in the formula at equal amounts at 6-10 percent by volume

12. The method of claim 10, wherein deionized water is from about 60 to about 75 percent of the formulation by volume.

13. The method of claim 12, wherein ethyl alcohol is from about 20 to about 30 percent of the formulation by volume.

14. The method of claim 13, wherein the oil extracts are from about 6 to 10 percent of the formulation in equal amounts by volume.
